(19) United States
(12) Patent Application Publication
(10) Pub. No.: US 2016/0271056 A1
(43) Pub. Date: Sep. 22, 2016

(54) CYSTIC FIBROSIS TREATMENT COMPRISING NITRIC OXIDE

(71) Applicants: NITRIC SOLUTIONS INC., Alberta (CA); 12TH MAN TECHNOLOGIES, INC., Garden Grove, CA (US)

(72) Inventors: Christopher Miller, North Vancouver (CA); Gilly Regev, Vancouver (CA); Alex Stenzler, Long Beach, CA (US)

(73) Assignees: Nitric Solutions Inc., Alberta, BC (CA); 12th Man Technologies, Inc., Garden Grove, CA (US)

(21) Appl. No.: 15/035,898

(22) PCT Filed: Dec. 11, 2014
(86) PCT No.: PCT/US2014/069806

§ 371 Date: May 11, 2016

Related U.S. Application Data
(60) Provisional application No. 61/920,219, filed on Dec. 23, 2013.

(51) Int. Cl. A61K 9/00; A61M 16/12; A61K 33/00

(52) U.S. Cl. CPC A61K 9/0073; A61K 33/00; A61M 16/12

(57) ABSTRACT

One aspect of the invention provides a method of treating a subject diagnosed with cystic fibrosis, but not having a pathological bacterial colonization in either lung. The method includes administering gaseous nitric oxide at a concentration of between about 40 ppm and about 400 ppm to the subject's lungs wherein said nitric oxide is administered by inhalation, and further wherein said nitric oxide induces conformational change in the cystic fibrosis transmembrane conductance regulator (CFTR) in the cells of the lung of the subject. Another aspect of the invention provides a kit comprising: an apparatus for administering gaseous nitric oxide to a subject and instructions for administering the gaseous nitric oxide according to one or more of the methods described herein.

### CROSS-REFERENCE TO RELATED APPLICATIONS

This application claims priority to U.S. Provisional Patent Application Ser. No. 61/920,219, filed Dec. 23, 2013. The entire content of this application is hereby incorporated by reference herein.

### BACKGROUND

The basic genetic defect in cystic fibrosis (CF) is altered function and/or lack of expression of the cystic fibrosis transmembrane conductance regulator (CFTR) protein. Dysfunction of this cAMP-activated ion channel protein leads to abnormal salt and water in epithelial cells of various tissues. In the lung, these changes incite alterations in composition and quality of the periciliary airway surface liquid, impairing airway defenses and leading to mucus obstruction, infection and inflammation.

### SUMMARY OF THE INVENTION

One aspect of the invention provides a method of treating a subject diagnosed with cystic fibrosis, but not having a pathological bacterial colonization in either lung. The method includes administering gaseous nitric oxide at a concentration of between about 40 ppm and about 400 ppm to the subject's lungs. The nitric oxide is administered by inhalation. The nitric oxide induces conformational change in the cystic fibrosis transmembrane conductance regulator (CFTR) in the cells of the lung of the subject.

This aspect of the invention can have a variety of embodiments. The subject can be a human.

The gaseous nitric oxide can be administered at a concentration between about 40 ppm and about 120 ppm. The gaseous nitric oxide can be administered at a concentration between about 120 ppm and about 200 ppm. The gaseous nitric oxide can be administered at a concentration between about 120 ppm and about 160 ppm. The gaseous nitric oxide can be administered at a concentration between about 160 ppm and about 200 ppm. The gaseous nitric oxide can be administered at a concentration between about 200 ppm and about 300 ppm. The gaseous nitric oxide can be administered at a concentration between about 300 ppm and about 400 ppm.

The gaseous nitric oxide can be administered at a concentration selected from the group consisting of: between about 40 ppm and about 50 ppm, between about 50 ppm and about 60 ppm, between about 60 ppm and about 70 ppm, between about 70 ppm and about 80 ppm, between about 80 ppm and about 90 ppm, between about 90 ppm and about 100 ppm, between about 100 ppm and about 110 ppm, between about 110 ppm and about 120 ppm, between about 120 ppm and about 130 ppm, between about 130 ppm and about 140 ppm, between about 140 ppm and about 150 ppm, between about 150 ppm and about 160 ppm, between about 160 ppm and about 170 ppm, between about 170 ppm and about 180 ppm, between about 180 ppm and about 190 ppm, between about 190 ppm and about 200 ppm, between about 200 ppm and about 210 ppm, between about 210 ppm and about 220 ppm, between about 220 ppm and about 230 ppm, between about 230 ppm and about 240 ppm, between about 240 ppm and about 250 ppm, between about 250 ppm and about 260 ppm, between about 260 ppm and about 270 ppm, between about 270 ppm and about 280 ppm, between about 280 ppm and about 290 ppm, between about 290 ppm and about 300 ppm, between about 300 ppm and about 310 ppm, between about 310 ppm and about 320 ppm, between about 320 ppm and about 330 ppm, between about 330 ppm and about 340 ppm, between about 340 ppm and about 350 ppm, between about 350 ppm and about 360 ppm, between about 360 ppm and about 370 ppm, between about 370 ppm and about 380 ppm, between about 380 ppm and about 390 ppm, and between about 390 ppm and about 400 ppm.

The gaseous nitric oxide can be administered for between about 1 minute and about 60 minutes.

The gaseous nitric oxide can be administered for a period selected from the group consisting of: between about 1 minute and about 5 minutes, between about 5 minutes and about 10 minutes, between about 10 minutes and about 15 minutes, between about 15 minutes and about 20 minutes, between about 20 minutes and about 25 minutes, between about 25 minutes and about 30 minutes, between about 30 minutes and about 35 minutes, between about 35 minutes and about 40 minutes, between about 40 minutes and about 45 minutes, between about 45 minutes and about 50 minutes, between about 50 minutes and about 55 minutes, and between about 55 minutes and about 60 minutes.

The gaseous nitric oxide can be administered for about 30 minutes.

The administering step can be performed between 1 and 5 times per day. The administering step can be performed between 1 and 3 times per day. The administering step can be performed between 1 and 7 days per week.

The gaseous nitric oxide can be administered until a target methemoglobin (metHb) level is achieved in the subject. The target methemoglobin (metHb) level can be between about 2% and about 5%.

Another aspect of the invention provides a kit including: an apparatus for administering gaseous nitric oxide to a subject; and instructions for administering the gaseous nitric oxide according to the methods described herein.

This aspect of the invention can have a variety of embodiments. The kit can further include a nitric oxide source. The nitric oxide source can be a tank of nitric oxide. The nitric oxide source can be a nitric oxide generator.

### BRIEF DESCRIPTION OF THE DRAWINGS

For a fuller understanding of the nature and desired objects of the present invention, reference is made to the following detailed description taken in conjunction with the figure wherein:

FIG. 1 depicts a method of treating subject diagnosed with cystic fibrosis, but not having bacterial colonization in their airway according to an embodiment of the invention; and

FIG. 2 depicts a kit 200 according to an embodiment of the invention.

### DEFINITIONS

The instant invention is most clearly understood with reference to the following definitions.

As used in the specification and claims, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

Unless specifically stated or obvious from context, as used herein, the term “or” is understood to be inclusive.

### DESCRIPTION OF THE INVENTION

Various aspects of the invention provide methods and kits for treating a subject diagnosed with cystic fibrosis, but not having a pathological bacterial colonization in their airway. The methods and kits described herein can advantageously correct an abnormally functioning CFTR protein. Such methods and kits can be used, for example, by or for individuals such as infants, children, and young adults diagnosed with cystic fibrosis, but who have not developed an airway colonization. Additionally, such methods and kits can be used prophylactically in individuals diagnosed with CF after an airway colonization is treated in order to prevent or minimize likelihood of a future airway colonization.

Various attempts to replace the CF gene with gene therapy have been unsuccessful. Included among these failed attempts are drugs known to improve the function called “potentiators” and “correctors”. Ivacaftor (KALYDECO®) is one such potentiator delivered as an oral medication. Correctors are compounds aimed at increasing the quantity of functional CFTR protein at the apical surface. The mechanistic pathway for some correctors is to stimulate the maturation of the CFTR protein by increasing levels of the messenger molecule S-nitrosothiol (GSNO). One pathway that reduces GSNO is GSNO reductase. An experimental corrector agent under investigation is a GSNO-reductase-reducing agent (inhibitor) that, therefore, preserves GSNO. These are typically delivered by intravenous means with a potential transition to oral preparations in the future.

There are at least three ways to increase GSNO in the airway cells. One way as described above is to deliver GSNO reductase inhibitors that prevent the reduction in GSNO levels. An alternative approach involves administering L-arginine, which is a precursor molecule that may result in increased NO if nitric oxide synthesases and other factors are present. The increased NO can then combine with intracellular thiols to form GSNO. Aspects of the invention propose a third approach, namely, increasing the levels of GSNO by the addition of nitric oxide directly to the cells, where it combines with intracellular thiols to form GSNO.

Embodiments of the invention increase the level of S-nitrosothiol (GSNO) at the airway apical surface by the delivery of nitric oxide gas to the airway. Nitric oxide gas is the smallest signaling molecule in the body. It is lipophilic and easily passes through the cell walls of most body cells. Once inside the cell, NO binds with glutathione to form GSNO directly.

Embodiments of the invention advantageously provide the NO directly to the airway surface cells as a gas, which promotes a uniform distribution of NO within the airway independent of blood flow. Additionally, better dose control is possible by modulating the concentration of NO administered to the airway cells. Moreover, embodiments of the invention have a better safety profile than other therapies because the inhaled NO is converted to methemoglobin and into nitrates once it enters the pulmonary vascular bed and therefore does not have downstream physiologic effects.

The invention includes a method for correcting the CFTR protein by the delivery of inhaled nitric oxide in concentrations between 40 and 400 ppm (preferably between 120 and 200 ppm) for approximately 30 minutes (although the time can vary), between 1 and 5 times (preferably between 1 and 3 times) a day (although the number of times per day can vary).

Methods of Treating a Subject Diagnosed with Cystic Fibrosis

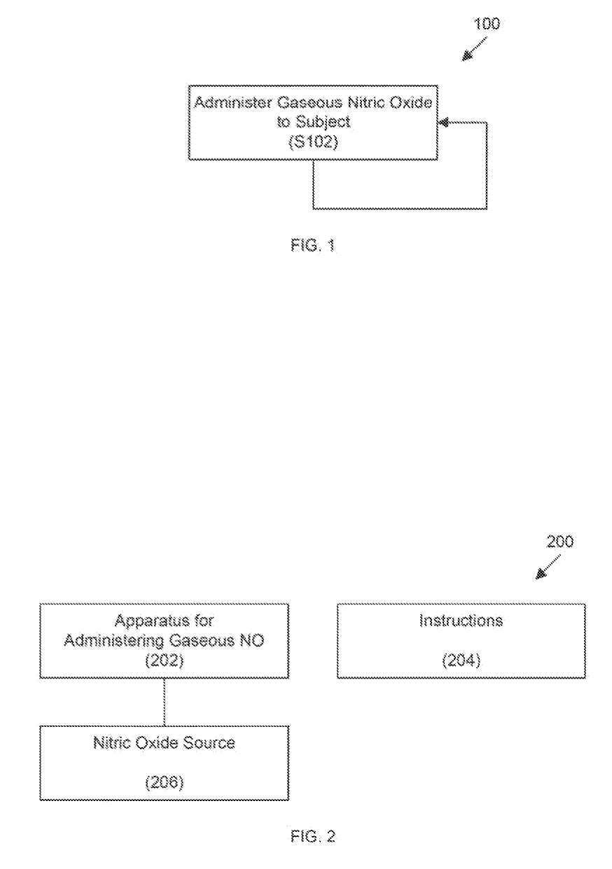

Referring now to FIG. 1, a method 100 of treating subject diagnosed with cystic fibrosis, but not having bacterial colonization in their airway is provided.

In step 102, gaseous nitric oxide is administered to the subject. The gaseous NO can be administered at a concentration of between about 40 ppm and about 400 ppm to the subject's lungs. The NO can be administered by inhalation from a device for administering a gas including those apparatuses discussed herein. The NO can induce conformational changes in the cystic fibrosis transmembrane conductance regulator (CFTR) in the cells of the lung of the subject.

The gaseous nitric oxide can be administered at a sufficient concentration, durations, and frequencies in order to induce conformational changes in the cystic fibrosis transmembrane conductance regulator (CFTR) in the cells of the airway of the subject. Such concentrations, durations, and frequencies can be optimized by a medical professional based on feedback from the subject, clinical trials, regulatory approvals, and the like.

Exemplary concentration ranges include, but are not limited to: between about 40 ppm and about 120 ppm, between about 120 ppm and about 200 ppm, between about 120 ppm and about 160 ppm, between about 160 ppm and about 200 ppm, between about 200 ppm and about 300 ppm, between about 300 ppm and about 400 ppm, between about 40 ppm and about 50 ppm, between about 50 ppm and about 60 ppm, between about 60 ppm and about 70 ppm, between about 70 ppm and about 80 ppm, between about 80 ppm and about 90 ppm, between about 90 ppm and about 100 ppm, between about 100 ppm and about 110 ppm, between about 110 ppm and about 120 ppm, between about 120 ppm and about 130 ppm, between about 130 ppm and about 140 ppm, between about 140 ppm and about 150 ppm, between about 150 ppm and about 160 ppm, between about 160 ppm and about 170 ppm, between about 170 ppm and about 180 ppm, between about 180 ppm and about 190 ppm, between about 190 ppm and about 200 ppm, between about 200 ppm and about 210 ppm, between about 210 ppm and about 220 ppm, between about 220 ppm and about 230 ppm, between about 230 ppm and about 240 ppm, between about 240 ppm and about 250 ppm, between about 250 ppm and about 260 ppm, between about 260 ppm and about 270 ppm, between about 270 ppm and about 280 ppm, between about 280 ppm and about 290 ppm, between about 290 ppm and about 300 ppm, between about 300 ppm and about 310 ppm, between about 310 ppm and about 320 ppm, between about 320 ppm and about 330 ppm, between about 330 ppm and about 340 ppm, between about 340 ppm and about 350 ppm, between about 350 ppm and about 360 ppm, between about 360 ppm and about 370 ppm, between about 370 ppm and about 380 ppm, between about 380 ppm and about 390 ppm, and between about 390 ppm and about 400 ppm.

Concentrations expressed herein can be with respect to air (e.g., a mixture including gaseous nitrogen and gaseous oxygen), which can be introduced by an apparatus for administering the NO.

Exemplary durations of treatments include between about 1 minute and about 60 minutes, between about 1 minute and about 5 minutes, between about 5 minutes and about 10 minutes, between about 10 minutes and about 15 minutes, between about 15 minutes and about 20 minutes, between about 20 minutes and about 25 minutes, between about 25 minutes and about 30 minutes, between about 30 minutes and about 35 minutes, between about 35 minutes and about 40 minutes, between about 40 minutes and about 45 minutes, between about 45 minutes and about 50 minutes, between about 50 minutes and about 55 minutes, between about 55 minutes and about 60 minutes, and about 30 minutes.

Additionally or alternatively, treatments can extend until a target methemoglobin (metHb) level is achieved in the subject. Examples of suitable target methemoglobin (metHb) levels include: between about 2% and about 5%, between about 2% and about 3%, between about 3% and about 4%, between about 4% and about 5%, and the like.

MetHb levels can be measured using a variety of techniques and apparatuses. For example, metHb levels can be measured using a blood sample (venous or arterial) using a photospectrometer. Examples of photospectrometers include the ABL 730 and OSM3 models available from Radiometer America Inc. of Westlake, Ohio. In another example, a pulse oximeter can be used to noninvasively measure metHb using light waveforms transmitted through a subject's finger. Exemplary pulse oximeters include the Masimo Rad-57 available from Masimo Corporation of Irvine, Calif.

Exemplary frequencies include between 1 and 5 times per day and between 1 and 3 times per day.

Gaseous NO and the associated air can be administered at any temperature that is comfortable and safe for the subject. Such temperatures include room temperature (21° C.), temperatures below room temperature (e.g., between about 10° C. and about 15° C. or between about 15° C. and about 21° C.) or above room temperature (e.g., between about 21° C. and about 25° C., between about 25° C. and about 30° C., between about 30° C. and about 35° C., and between about 35° C. and about 40° C.).

Gaseous NO and the associated air can be administered at any humidity that is comfortable and safe for the subject.

Method 100 can be performed in a medical setting such as a hospital, a clinic, a nursing home, and the like. Method 100 can be performed by medical professional. Additionally or alternatively, method 100 can be performed by a subject or a subject's caregiver in a non-medical setting such as a home, a school, or a workplace.

### Kits for Administering Gaseous Nitric Oxide

Referring now to FIG. 2, a kit 200 is provided including an apparatus for administering gaseous nitric oxide 202 and instructions 204 for administering the gaseous nitric oxide according to the methods described herein.

Apparatus 202 can include one or more systems for interfacing with the subject's mouth and/or nose and one or more systems for detecting and/or controlling the concentration of NO administered to the subject. Various apparatus 202 for administering NO are described in publications such as U.S. Pat. Nos. 5,558,083; 6,581,599; 6,668,828; 6,786,217; 6,955,171; 6,986,351; 7,255,105; and 7,516,742 as well as International Publication No. WO 2012/075420.

Apparatus 202 can be coupled to a nitric oxide source 206, for example, a tank of nitric oxide, a nitric oxide supply line, and/or a nitric oxide generator. Various nitric oxide generators are described in U.S. Pat. Nos. 5,396,882 and 8,574,531 and U.S. Patent Application Publication Nos. 2013/0037023.

### EQUIVALENTS

Those skilled in the art will recognize, or be able to ascertain using no more than routine experimentation, many equivalents of the specific embodiments of the invention described herein. Such equivalents are intended to be encompassed by the following claims.

### INCORPORATION BY REFERENCE

The entire contents of all patents, published patent applications, and other references cited herein are hereby expressly incorporated herein in their entireties by reference.

1. A method of treating a subject diagnosed with cystic fibrosis, but not having a pathological bacterial colonization in either lung, the method comprising:
administering gaseous nitric oxide at a concentration of between about 40 ppm and about 400 ppm to the subject's lungs;
wherein the nitric oxide is administered by inhalation; and
further wherein the nitric oxide induces conformational change in the cystic fibrosis transmembrane conductance regulator (CFTR) in the cells of the lung of the subject.

2. The method of claim 1, wherein the subject is a human.

3. The method of claim 1, wherein the gaseous nitric oxide is administered at a concentration between about 40 ppm and about 120 ppm.

4. The method of claim 1, wherein the gaseous nitric oxide is administered at a concentration between about 120 ppm and about 200 ppm.

5. The method of claim 1, wherein the gaseous nitric oxide is administered at a concentration between about 120 ppm and about 160 ppm.

6. The method of claim 1, wherein the gaseous nitric oxide is administered at a concentration between about 160 ppm and about 200 ppm.

7. The method of claim 1, wherein the gaseous nitric oxide is administered at a concentration between about 200 ppm and about 300 ppm.

8. The method of claim 1, wherein the gaseous nitric oxide is administered at a concentration between about 300 ppm and about 400 ppm.

9. The method of claim 1, wherein the gaseous nitric oxide is administered at a concentration selected from the group consisting of: between about 40 ppm and about 50 ppm, between about 50 ppm and about 60 ppm, between about 60 ppm and about 70 ppm, between about 70 ppm and about 80 ppm, between about 80 ppm and about 90 ppm, between about 90 ppm and about 100 ppm, between about 100 ppm and about 110 ppm, between about 110 ppm and about 120 ppm, between about 120 ppm and about 130 ppm, between about 130 ppm and about 140 ppm, between about 140 ppm and about 150 ppm, between about 150 ppm and about 160 ppm, between about 160 ppm and about 170 ppm, between about 170 ppm and about 180 ppm, between about 180 ppm and about 190 ppm, between about 190 ppm and about 200 ppm, between about 200 ppm and about 210 ppm, between about 210 ppm and about 220 ppm, between about 220 ppm and about 230 ppm, between about 230 ppm and about 240 ppm, between about 240 ppm and about 250 ppm, between about 250 ppm and about 260 ppm, between about 260 ppm and about 270 ppm, between about 270 ppm and about 280 ppm, between about 280 ppm and about 290 ppm, between about 290 ppm and about 300 ppm, between about 300 ppm and about 310 ppm, between about 310 ppm and about 320 ppm, between about 320 ppm and about 330 ppm, between about 330 ppm and about 340 ppm, between about 340 ppm and about 350 ppm, between about 350 ppm and about 360 ppm, between about 360 ppm and about 370 ppm, between about 370 ppm and about 380 ppm, between about 380 ppm and about 390 ppm, and between about 390 ppm and about 400 ppm.

10. The method of claim 1, wherein the gaseous nitric oxide is administered for between about 1 minute and about 60 minutes.

11. The method of claim 1, wherein the gaseous nitric oxide is administered for a period selected from the group consisting of: between about 1 minute and about 5 minutes, between about 5 minutes and about 10 minutes, between about 10 minutes and about 15 minutes, between about 15 minutes and about 20 minutes, between about 20 minutes and about 25 minutes, between about 25 minutes and about 30 minutes, between about 30 minutes and about 35 minutes, between about 35 minutes and about 40 minutes, between about 40 minutes and about 45 minutes, between about 45 minutes and about 50 minutes, between about 50 minutes and about 55 minutes, and between about 55 minutes and about 60 minutes.

12. The method of claim 1, wherein the gaseous nitric oxide is administered for about 30 minutes.

13. The method of claim 1, wherein the administering step is performed between 1 and 5 times per day.

14. The method of claim 1, wherein the administering step is performed between 1 and 3 times per day.

15. The method of claim 1, wherein the administering step is performed between 1 and 7 days per week.

16. The method of claim 1, wherein the gaseous nitric oxide is administered until a target methemoglobin (metHb) level is achieved in the subject.

17. The method of claim 16, wherein the target methemoglobin (metHb) level is between about 2% and about 5%.

18. A kit comprising:
an apparatus for administering gaseous nitric oxide to a subject; and
instructions for administering the gaseous nitric oxide according to the method of claim 1.

19. The kit of claim 18, further comprising:
a nitric oxide source.

20. The kit of claim 18, wherein the nitric oxide source is selected from the group consisting of: a tank of nitric oxide and a nitric oxide generator.

21. (canceled)
